(19) United States
(12) Patent Application Publication
(10) Pub. No.: US 2022/0117921 A1
(43) Pub. Date: Apr. 21, 2022

(54) PHARMACEUTICAL COMBINATIONS FOR THE TREATMENT OF PAIN

(71) Applicant: IMUNEKS FARMA ILAC SANAYI VE TICARET A.S., Istanbul (TR)

(72) Inventor: Mehmet Nevzat PISAK, Besiktas/Istanbul (TR)

(21) Appl. No.: 17/289,606

(22) PCT Filed: Oct. 16, 2019
(86) PCT No.: PCT/TR2019/050873

§ 371 Date: Apr. 28, 2021

(30) Foreign Application Priority Data
Nov. 1, 2018 (TR) 2018/16399

(51) Int. Cl. A61K 31/192; A61P 29/00; A61K 31/196; A61K 31/138

(52) U.S. Cl. CPC A61K 31/192; A61K 31/138; A61K 31/196; A61P 29/00

(57) ABSTRACT

The present invention relates to pharmaceutical combination for use in the treatment of pain comprising a beta blocker and an NSAID, pharmaceutical compositions comprising the combination and processes for preparation of the same. The present invention provides a synergistic drug combination comprising an NSAID or a pharmaceutically acceptable salt thereof and a beta blocker or a pharmaceutically acceptable salt thereof for use in the therapeutic treatment of pain, wherein the pain can be muscle pain, eye pain, orofacial pain, abdominal pain, gynecological pain, tissue or skin pain, inflammatory pain, pain resulting from inflammatory bowel disease, bone pain, back pain, gout pain, fibromylagia, allodynia, dysmenorrhea, headache, cluster headache, migraine pain, tendon pain, postoperative pain, post-stroke pain, cancer pain, dental pain, diabetic neuropathy, dysmenorrhea pain, stump pain, phantom pain, neuralgias, neuronitis, neuritis, AIDS-related neuropathy, MS-related neuropathy or spinal cord injury-related pain.

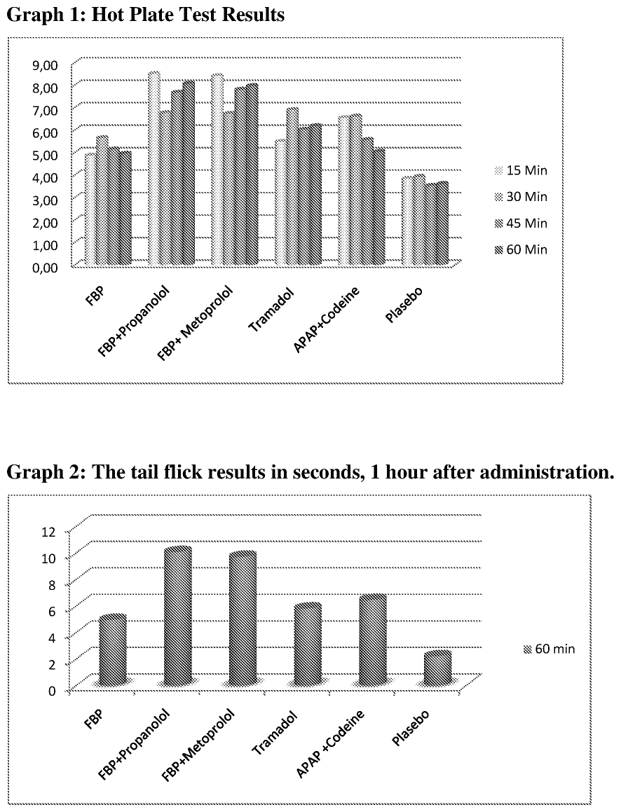

### TECHNICAL FIELD

The present invention relates to pharmaceutical combinations for use in the treatment of pain comprising a beta blocker and an NSAID, pharmaceutical compositions comprising the combination and processes for preparation of the same.

### BACKGROUND ART

Non-Steroidal Anti-Inflammatory Drugs (NSAIDs) are a diverse group of compounds that are mainly used for the treatment of mild to moderate pain. They are also used to reduce fever and inflammation. As being non-narcotic compared to opioids, NSAIDs are among the most frequently used classes of medications. They exert their pharmacological action by inhibiting the synthesis of prostaglandins (PGs) by non-selectively blocking cyclooxygenases 1 and 2 (COX-1 and COX-2) or by selectively blocking COX-2. Thus, the classification system of NSAIDs is based on their chemical structure and whether they inhibit the COX-1 and/or COX-2 enzymes.

Most used and well known combination of NSAIDs are with acetaminophen (paracetamol). For example, ibuprofen which is one of the most used NSAID can be combined with acetaminophen (paracetamol) for the treatment of pain as disclosed in the study of Merry et al. (Combined acetaminophen and ibuprofen for pain relief after oral surgery in adults: a randomized controlled trial, Br J Anaesth. 2010 January).

In addition, there have been binary combinations of NSAIDs and gastro protective agents in single unit dosage forms disclosed in the state of the art, in order to prevent or treat gastrointestinal side effects. US patent application US 2014186439 relates to a new stable pharmaceutical composition comprising the combination of ibuprofen and famotidine with at least one pharmaceutically acceptable excipient. US patent application US 20070237820 A1 relates to a solid oral dosage form comprising a first portion comprising an NSAID (preferably diclofenac); and a coating comprising an anti-ulcerative compound (preferably misoprostol). However, none of these prior arts provide or aim a higher efficient therapy.

US patent application; US20160022639A1 discloses the combination of NSAIDs with anti-hypertensive agents. But, the use of an anti-hypertensive agent in combination with an NSAID is for reducing the edema caused by the NSAID. The aforementioned patent application especially focuses on selective COX-2 inhibitors and the relief of edema caused by COX-2 selective NSAIDs rather than increasing the analgesic effect of the NSAID, furthermore it also discloses that beta-blockers were ineffective for the relief of edema. This patent application does not suggest a better analgesic effect of NSAID.

EP1865779A2 and EP1990049A2 disclose a combination of beta blockers with NSAIDs and several other anti hypertensive and cardio protective agents for the treatment of a maladaptive nutritional state secondary to a chronic Systemic Inflammatory Response State (SIRS), such as cachexia or anorexia but do not mention any effect of such a combination for the treatment of pain.

Therefore, there still exists a particular need for providing a therapeutic treatment of pain with a significant efficiency while reducing the side effects and providing a good safety profile.

### BRIEF DESCRIPTION OF THE INVENTION

The present invention provides a synergistic drug combination comprising an NSAID or a pharmaceutically acceptable salt thereof and a beta blocker or a pharmaceutically acceptable salt thereof for use in the therapeutic treatment of muscle pain, eye pain, orofacial pain, abdominal pain, gynecological pain, tissue or skin pain, inflammatory pain, pain resulting from inflammatory bowel disease, bone pain, back pain, gout pain, fibromylagia, allodynia, dysmenorrhea, headache, cluster headache, migraine pain, tendon pain, postoperative pain, post-stroke pain, cancer pain, dental pain, diabetic neuropathy, dysmenorrhea pain, stump pain, phantom pain, neuralgias, neuronitis, neuritis, AIDS-related neuropathy, MS-related neuropathy or spinal cord injury-related pain.

In another aspect, the present invention provides a combination for use in the therapeutic treatment of muscle pain, back pain, post-operative pain, headache, dysmenorrhea, cluster headache, cancer pain or migraine pain.

In a further aspect, the present invention provides a combination comprising a beta blocker and an NSAID, wherein NSAID is preferably selected from non-selective NSAIDs which are ibuprofen, flurbiprofen, diclofenac, naproxen, meloxicam, piroxicam, acetyl salicylic acid, ketoprofen, dexketoprofen, ketorolac, lornoxicam, tenoxicam and pharmaceutically acceptable salts and/or derivatives thereof; more preferably flurbiprofen, ibuprofen, diclofenac, dexketoprofen, piroxicam, tenoxicam, naproxen, meloxicam and pharmaceutically acceptable salts and/or derivatives thereof.

In another aspect, the present invention provides a combination comprising a beta blocker and an NSAID wherein beta blocker is selected from the group consisting of acebutolol, atenolol, betaxolol, propranolol, bisoprolol, celiprolol, esmolol, metoprolol, nebivolol and pharmaceutically acceptable salts and/or derivatives thereof; preferably metoprolol, propranolol, nebivolol, atenolol, betaxolol, bisoprolol, carvedilol and pharmaceutically acceptable salts and/or derivatives thereof.

In another aspect, the present invention provides a pharmaceutical combination for the therapeutic treatment of pain including moderate to severe pain without any addiction to the medicament of the present invention.

In further aspect, the present invention relates to a pharmaceutical combination providing an effective treatment of pain with a reduced side effect profile compared to a treatment with an opioid receptor agonist with a large number of side effects and also providing a surprisingly superior level of analgesic effects compared to the combinations found in the prior art.

### DETAILED DESCRIPTION OF THE INVENTION

The present invention relates to a synergistic drug combination comprising an NSAID and a beta-blocker or a pharmaceutically acceptable salts or derivatives thereof for use in the therapeutic treatment of pain. It has been surprisingly found that when a beta blocker is combined with an NSAID, beta blocker acts synergistically with the NSAID and produces a much greater reduction in pain than when the NSAID is administered alone.

As used herein, the term “therapeutic treatment” means treatment for a subject already having the disease, by administering to the subject the therapeutically effective amount of the composition of the present invention. Thus, the present invention is related to the therapeutic treatment and relief of pain while the pain is already present in the subject who is to be administered the drug combination or the composition of the present invention.

The term “treatment” as used herein essentially means the cure, amelioration or improvement of a disease, pathological condition or disorder.

The term “pharmaceutically acceptable salt” as used herein essentially means the organic or inorganic salts of the active ingredient, in that case NSAID or beta blocker, including inorganic or organic acid addition salts of the active ingredient, exhibiting minimal or no undesired toxicological effects.

The term “derivative(s)” as used herein essentially means a compound which is formed by a chemical process from a parent compound, wherein the chemical formula of the parent compound is present in the derivative.

The term “daily dose” or “daily amount” as used herein essentially means the effective amount (dose) of NSAID and beta blocker administered simultaneously, concomitantly or as a fixed unit dosage form to a patient, within a period of 24 hours, for the therapeutic treatment of pain.

The term “subject” or “patient” as used herein is equivalent to a mammal in need of a treatment for a pain and could be a living human or an animal such as cat, dog and cattle.

The pain treated with the use of the combination of the present invention may be moderate to severe pain and/or chronic or acute pain and/or nociceptive or neuropathic pain.

In one embodiment, the present invention provides a pharmaceutical combination for use in the therapeutic treatment of pain comprising;

a) an NSAID or pharmaceutically acceptable salts thereof; and
b) a beta blocker or pharmaceutically acceptable salts thereof,
wherein the pain is muscle pain, eye pain, orofacial pain, abdominal pain, gynecological pain, tissue or skin pain, inflammatory pain, pain resulting from inflammatory bowel disease, bone pain, back pain, gout pain, fibromylagia, allodynia, dysmenorrhea, headache, cluster headache, migraine pain, tendon pain, postoperative pain, post-stroke pain, cancer pain, dental pain, diabetic neuropathy, dysmenorrhea pain, stump pain, phantom pain, neuralgias, neuronitis, neuritis, AIDS-related neuropathy, MS-related neuropathy or spinal cord injury-related pain. More preferably, said pain is muscle pain, back pain, post-operative pain, headache, dysmenorrhea, cluster headache, cancer pain or migraine pain.

In one embodiment, said pain is preferably chronic pain or moderate to severe pain.

It has also been found that the combination of NSAIDs with beta blockers provide an efficient therapeutic treatment for these specific types of pains according to the present invention. As it is seen, some sort of pains which could not be treated with the use of NSAIDs alone, may be treated with the combination of the present invention and for other types of pains possible to be treated by NSAIDs, the combination provides a higher efficacy.

According to the present invention, beta blocker can be, but is not limited to, beta-1 selective beta blocker, beta-2 selective beta blocker, alpha-1/beta adrenergic antagonists, beta-3 selective beta blocker, beta-1 and beta-3 selective beta blocker and/or non-selective beta blocker (beta-1 and beta-2 selective beta-blocker or a mixture of two or more beta-blockers. Beta-1 selective beta blocker is selected from the group consisting of acebutolol, atenolol, betaxolol, bisoprolol, celiprolol, esmolol, metoprolol, nebivolol and pharmaceutically acceptable salts and derivatives thereof and their combinations. Non-selective beta blocker is selected from the group consisting of alprenolol, bucindolol, carteolol, levobunolol, medroxalol, mepindolol, metipranolol, nadolol, oxprenolol, penbutolol, pindolol, propafenone (propafenone is a sodium channel blocking drug that is also a beta-adrenergic receptor antagonist), propranolol, sotalol, timolol and pharmaceutically acceptable salts and derivatives thereof and their combinations. The beta blocker may also have an intrinsic sympathomimetic activity as acebutolol, betaxolol, carteolol, carvedilol, labetalol, oxprenolol, penbutolol, pindolol.

In one embodiment of the present invention, beta blocker is selected from the group consisting of acebutolol, atenolol, betaxolol, propranolol, bisoprolol, celiprolol, esmolol, metoprolol, nebivolol and pharmaceutically acceptable salts and/or derivatives thereof; preferably metoprolol, propranolol, nebivolol, atenolol, betaxolol, bisoprolol, carvedilol and pharmaceutically acceptable salts and/or derivatives thereof; more preferably metoprolol, propranolol and pharmaceutically acceptable salts and/or derivatives thereof.

The classification system of NSAIDs is based on their chemical structure and whether they inhibit the COX-1 and/or COX-2 enzymes: 2-Arylpropionic acids group, salicylates group, arylalkanoic acids group and oxicams group. Although there are more than these four groups, the preferred embodiment of the present invention is focused on these four groups of non-selective (equipotent) COX inhibitors based on their ability to inhibit both the COX-1 and COX-2 enzymes, and there is also a group classified as selective COX-2 inhibitors, which would not be ideally used within the combination of the present invention, due to the serious risk of cardiovascular side effects. Thus, not to limit the scope of the present invention, the preferred embodiment of the present invention would include the use of a non-selective COX inhibitor/NSAID rather than a COX-2 inhibitor NSAID which have serious cardiovascular side effects.

Non-selective NSAIDs have a completely different mechanism of action compared to opioid medication interacting with opioid receptors. By using NSAIDs, the treatment of moderate to severe pain is provided and the use of opioids with serious addiction properties is eradicated.

In one embodiment of the present invention, NSAID is selected from the group consisting of 2-Arylpropionic acids group including alminoprofen, benoxaprofencarprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid; salicylates group including alicylamide, salicyl salicylate, methyl salicylate, magnesium salicylate, faislamine, ethenzamide, diflunisal, choline magnesium salicylate, benorylate/benorilatem and amoxiprin, aspirin; arylalkanoic acids group including aceclofenac, acemetacin, alclofenac, bromfenac, diclofenac, etodolac, indometacin, nabumetone, oxametacin, proglumetacin, sulindac, and tolmetinor; and oxicams group including droxicam, lomoxicam, meloxicam, piroxicam, tenoxicam and pharmaceutically acceptable salts and derivatives thereof and their combinations.

In another embodiment of the present invention, NSAID is a non-selective COX inhibitor which is selected from the group consisting of ibuprofen, flurbiprofen, diclofenac, naproxen, meloxicam, piroxicam, acetyl salicylic acid, ketoprofen, dexketoprofen, ketorolac, lornoxicam, tenoxicam and pharmaceutically acceptable salts or derivatives thereof; preferably flurbiprofen, ibuprofen, diclofenac, dexketoprofen, piroxicam, tenoxicam, naproxen, meloxicam and more preferably flurbiprofen, ibuprofen, diclofenac, naproxen, meloxicam and pharmaceutically acceptable salts or derivatives thereof.

In one embodiment the pharmaceutical combination of the present invention comprises an NSAID or its pharmaceutically acceptable salt and/or derivatives thereof in a daily dose of between 3 to 3200 mg, preferably between 10 to 3200 mg and wherein when the NSAID is flurbiprofen, the daily dose is between 40 to 300 mg, when the NSAID is diclofenac, the daily dose is between 20 to 220 mg, when the NSAID is meloxicam, the daily dose is between 6 to 30 mg, when the NSAID is naproxen, the daily dose is between 200 to 1300 mg and when the NSAID is ibuprofen, the daily dose is between 400 to 3200 mg.

In one embodiment, the pharmaceutical combination of the present invention comprises a beta blocker or its pharmaceutically acceptable salt thereof wherein the daily dose of beta blocker is in the range of from 4 to 80, preferably 6 to 70, more preferably 8 to 60.

According to the present invention, the most preferred pharmaceutical combinations for use in the treatment of muscle pain, eye pain, orofacial pain, abdominal pain, gynecological pain, tissue or skin pain, inflammatory pain, pain resulting from inflammatory bowel disease, bone pain, back pain, gout pain, fibromylagia, allodynia, dysmenorrhea, headache, cluster headache, migraine pain, tendon pain, postoperative pain, post-stroke pain, cancer pain, dental pain, diabetic neuropathy, dysmenorrhea pain, stump pain, phantom pain, neuralgias, neuronitis, neuritis, AIDS-related neuropathy, MS-related neuropathy or spinal cord injury-related pain are as follows:

According to the present invention, the most preferred pharmaceutical combinations for use in the treatment of pain are as follows:

metoprolol in combination with flurbiprofen,
metoprolol in combination with naproxen,
propranolol in combination with flurbiprofen,
propranolol in combination with naproxen,
metoprolol in combination with diclofenac
metoprolol in combination with meloxicam,
propranolol in combination with diclofenac, or
propranolol in combination with meloxicam.

In another preferred embodiment, the present invention provides a therapeutic drug combination comprising an NSAID and a beta blocker for simultaneous administration or a pharmaceutical composition comprising the drug combination according to the present invention in a fixed unit dosage form for use in the therapeutic treatment of pain.

The term “fixed unit dosage form” as used herein means a single dosage unit comprising a combination of two or more active ingredients with pharmaceutically acceptable excipient(s), in that case an NSAID and a beta blocker.

The term “simultaneous” as used herein means that two active ingredients, in that case NSAID and beta blocker are administered at the same time and by the same route.

The term “concomitant administration” or “concomitantly” as used herein; means administration of a NSAID or beta blocker within a one hour time frame or when provided as a treatment package where both active compounds are given for administration in a single treatment package, provided in separate dosage units but inside the same pack for the treatment of pain.

In one embodiment of the present invention, a fixed unit dose pharmaceutical composition for use in the therapeutic treatment of pain is provided wherein the composition comprises a pharmaceutical combination which comprises;

a) an NSAID or pharmaceutically acceptable salts and/or derivatives thereof; and
b) a beta blocker or pharmaceutically acceptable salts and/or derivatives thereof,
wherein the pain is muscle pain, eye pain, orofacial pain, abdominal pain, gynecological pain, tissue or skin pain, inflammatory pain, pain resulting from inflammatory bowel disease, bone pain, back pain, gout pain, fibromylagia, allodynia, dysmenorrhea, headache, cluster headache, migraine pain, tendon pain, postoperative pain, post-stroke pain, cancer pain, dental pain, diabetic neuropathy, dysmenorrhea pain, stump pain, phantom pain, neuralgias, neuronitis, neuritis, AIDS-related neuropathy, MS-related neuropathy or spinal cord injury-related pain; and preferably the pain is muscle pain, back pain, post-operative pain, headache, dysmenorrhea, cluster headache, cancer pain or migraine pain.

In one embodiment of the present invention, a fixed unit dose pharmaceutical composition for use in the therapeutic treatment of pain is provided wherein the composition comprises a pharmaceutical combination which comprises;

a) an NSAID or pharmaceutically acceptable salts and/or derivatives thereof;
b) a beta blocker or pharmaceutically acceptable salts and/or derivatives thereof, and
c) at least one pharmaceutically acceptable excipient,
wherein the pain is muscle pain, eye pain, orofacial pain, abdominal pain, gynecological pain, tissue or skin pain, inflammatory pain, pain resulting from inflammatory bowel disease, bone pain, back pain, gout pain, fibromylagia, allodynia, dysmenorrhea, headache, cluster headache, migraine pain, tendon pain, postoperative pain, post-stroke pain, cancer pain, dental pain, diabetic neuropathy, dysmenorrhea pain, stump pain, phantom pain, neuralgias, neuronitis, neuritis, AIDS-related neuropathy, MS-related neuropathy or spinal cord injury-related pain.

In one embodiment of the present invention, the fixed unit dosage form may be an oral dosage form. This oral dosage form may be tablet, capsule, gel capsule, pellet, granule, tablet in tablet, tablet in capsule, powder, cachet, troche, caplet or coated tablet, preferably being tablet, capsule or powder. The pharmaceutical composition may also be in the form of drop, pellet, granule, aerosol, mouth wash, gargle, inhalable particle, inhalable solution, emulsion, lotion, solution, suspension, syrup, powder, transdermal patch, impregnated dressing, cream, ointment, gel, foam, paste, spray, suppository, ocular and injectable. Furthermore, the pharmaceutical composition may comprise pulsatile, sustained, delayed or controlled release, immediate or modified release mode of action.

The injectable dosage form may be powder to be mixed with water or another solvent for injection or liquid for injection and the dosage form can be administered as IM, IV and subcutaneously and preferably as IV.

The transdermal dosage form can be a topical preparation or a patch that can deliver the active ingredients of the composition to the blood stream of the subject.

In another embodiment of the present invention, the pharmaceutical composition further comprises at least one pharmaceutically acceptable excipient such as a carrier. Fixed dosage forms of the present invention may comprise suitable solvents, water, fillers, diluents, binders, lubricants, disintegrating agents, surfactants, glidants, sweetening agents, disaggregators, coloring agents and coating agents as pharmaceutically acceptable excipients and preferably disintegrant, lubricant and mixture thereof.

Pharmaceutically acceptable diluents of the present invention may be selected from magnesium stearate, lactose, microcrystalline cellulose, starch, pre-gelatinized starch, calcium phosphate, calcium sulphate, calcium carbonate, sodium starch glycolate, mannitol, sorbitol, xylitol, sucrose, maltose, fructose, dextrose and the like or mixtures thereof.

Pharmaceutically acceptable binders of the invention may be selected from starches, natural sugars, corn sweeteners, natural and synthetic gums, cellulose derivatives such as hydroxypropyl cellulose, hydroxypropyl methylcellulose, microcrystalline cellulose, gelatin, polyvinylpyrrolidone, polyethylene glycol, waxes, sodium alginate, alcohols, water and the like or mixtures thereof

Pharmaceutically acceptable lubricants of the present invention may be selected from metallic stearates, metallic lauryl sulfates, fatty acids, fatty acid esters, fatty alcohols, paraffins, hydrogenated vegetable oils, polyethylene glycols, boric acid, polyvinylpyrrolidone, sodium benzoate, sodium acetate, sodium chloride, talk and the like or mixtures thereof; preferably metallic stearates or fatty acid esters including glyceryl monostearate, glyceryl tribehenate, glyceryl dibehenate and glyceryl stearate are used.

Pharmaceutically acceptable disintegrating agents of the present invention may be selected from starches, cellulose derivatives, polyvinylpyrrolidone, crospovidone, clays, ion-exchange resins, alginic acid, sodium alginate and the like or mixtures thereof;

Pharmaceutically acceptable surfactants of the present invention may be selected from sulfates, sulfonates, phosphates, carboxylates, primary-secondary-tertiary amines, quaternary ammonium compounds, fatty alcohols, sugar esters of fatty acids, glycerides of fatty acids, polyoxy ethylene glycol alkyl ethers, polisorbates, sorbitan alkyl esters, poloxamers and the like or mixtures thereof.

Pharmaceutically acceptable glidants of the present invention may be selected from silicon dioxide, magnesium trisilicate, starch, talc, colloidal silicon dioxide, silicon hydrogel and the like or mixtures thereof.

### EXAMPLES

The present invention will be explained more in detail by illustrating Examples and study.

### Example 1

The following composition provides the contents of an embodiment of the present invention. The composition is in the form of a film tablet comprising flurbiprofen and propranolol hydrochloride.

Flurbiprofen/Propranolol hydrochloride 100 mg/10 mg Film Tablet

| Unit Formula | |
|---|---|
| Ingredients | mg/tb. |
| Flurbiprofen | 100.00 |
| Propranolol hydrochloride | 10.00 |
| Hydroxypropyl cellulose | 20.00 |
| Microcrystalline Cellulose type 101 | 100.00 |
| Crospovidone | 40.00 |
| Microcrystalline Cellulose type 102 | 40.00 |
| Colloidal silicon dioxide | 2.50 |
| Magnesium Stearate | 5.00 |
| Weight of Core Tablet | 327.50 |
| Aquarius ™ Preferred HSP Orange | 15 |
| Weight of Coated Tablet | 332.50 |

### Manufacturing Directions (Wet Granulation)

1. Sieve together Flurbiprofen, Propranolol, microcrystalline cellulose type 101 and hydroxypropylcellulose,
2. Granulate the mixture of step 1 with granulation solution prepared by distillated water and crospovidone (15 mg) in high shear granulator
3. Dry the mixture in fluid-bed granulator and pass through a 0.8-mm sieve,
4. Add microcrystalline cellulose type 102, crospovidone (25 mg) and mix 15 minutes.
5. Add magnesium stearate to the mixture and mix 3 minutes.
6. Compress tablets,
7. Coat tablets with coating solution (Preferred HSP orange)

Diclofenac Potassium/Metoprolol tartrate 50 mg/10 mg Film Tablet

| Unit Formula | |
|---|---|
| Ingredients | mg/tb. |
| Diclofenac potassium | 50.00 |
| Metoprolol tartrate | 10.00 |
| Lactose Granule | 28.00 |
| Microcrystalline Cellulose type 102 | 67.00 |
| Hydroxypropyl cellulose | 8.00 |
| Crospovidone | 10.00 |
| Colloidal silicon dioxide | 2.00 |
| Magnesium Stearate | 2.00 |
| Weight of Core Tablet | 177.00 |
| Aquarius ™ Preferred HSP BPP316041 Green | 7 |
| Weight of Coated Tablet | 184.00 |

### Manufacturing Directions (Direct Compression)

1. Sieve Diclofenac potassium, lactose granule, hydroxypropyl cellulose and mix 10 min
2. Add Metoprolol and microcrystalline cellulose and mix 10 min
3. Add crospovidone and mix 5 min
4. Sieve colloidal silicone dioxide and microcrystalline cellulose together and mix 5 min
5. Add magnesium stearate to the mixture and mix 3 minutes.
6. Compress tablets,
7. Coat tablets with coating solution (Preferred HSP BPP316041 Green)

### Pivotal Hot Plate Test Study

Flurbiprofen, propranolol and metoprolol are all well-established drugs from their respective classes and were used in this study on purpose to test the analgesic effect of the combination of a non selective COX inhibitor (flurbiprofen) with non-selective (propranolol) or beta-1 selective beta blocker (metoprolol).

Hot plate test of Eddy and Leimbach (1953) is a test of the pain response in animals and are well described in the study of Michael Williams, Roger D. Porsolt, in xPharm: The Comprehensive Pharmacology Reference, 2007.

In this study, a total of 42, 250±20 g in weight female Albino Wistar rats were used. The rats, were clinically healthy and they were fed ad libitum commercial rat feed and allowed to reach sterile reverse osmosis filtered water supply. Female rats were preferred as their higher degree nociceptive pain response than males (Craft et al. European Journal of Pain 8, 2004, 397-411). The animals were kept in polycarbonate universal rat cages. 12 hours light and 12 hours dark cycle were applied and environmental temperature was adjusted to 22±1° C. before the administration of the drugs as detailed below.

Drug and drug combinations in oral fixed dose powder form were diluted with distilled water to be administered by gavage to all the experimental groups. Treatment plan and drug doses were applied as indicated below.

TABLE 2
Treatment groups

| | | Drug and/or Drug Combinations as | | |
|---|---|---|---|---|
| Group | Rat | mg/kg with Hydroxypropyl cellulose | | |
| Name | Number | (HPMC) as carrier (excipient) | | |
| FBP | 7 | Flurbiprofen | | HPMC |
| (Flurbiprofen) | | 40 mg/kg | | 20 mg/kg |
| FBP + | 7 | Flurbiprofen | Propranolol | HPMC |
| Propranolol | | 40 mg/kg | 4 mg/kg | 20 mg/kg |
| FBP + | 7 | Flurbiprofen | Metoprolol | HPMC |
| Metoprolol | | 40 mg/kg | 5 mg/kg | 20 mg/kg |
| Tramadol | 7 | Tramadol | — | HPMC |
| | | 25 mg/kg | | 20 mg/kg |
| Paracetamol | 7 | Paracetamol | Codein | HPMC |
| (APAP) + | | 200mg/kg | 20 mg/kg | 20 mg/kg |
| Codeine | | | | |
| Placebo | 7 | HPMC | — | |
| | | 30 mg/kg | | |

In the experiment, the compositions at the doses specified in Table 3 are administered to all 6 groups with 7 rats in each through oral gavage.

### Analgesic Effect

For the evaluation of pain relieving efficacy of the tested drugs, heat induced pain model was used. In this rat model; the analgesic activity was evaluated in rats by using hot plate (Analgesic hot plate, May AHP603) maintained at 52.5° C.±0.1° C. Each rat was placed on the bottom of the hot plate surrounded by 20 cm diameter round plastic chamber. The touch of the footpads of the animal was accepted as the starting time and equipped chronometer recording was synchronously started. Hot plate test ended by the observation of pain related behaviors such as paw licking, limb flicking and/or jumping and then heat induced pain tolerance time of each animal was calculated. Hot plate test was repeated at four different timepoints following drug or placebo administration at 15 min, 30 min, 45 min and 60 min. The total latency per group was divided to 7 to get an average latency in seconds per group. A cut-off time of 20 seconds was used to avoid tissue injury. Graph 1 also shows the result of hot plate test demonstrating the latency in seconds.

TABLE 3
The results of the hot plate test demonstrating the latency in seconds

| | | FBP + | FBP + | | APAP + | |
|---|---|---|---|---|---|---|
| | FBP | Propanolol | Metoprolol | Tramadol | Codeine | Placebo |
| 15 Min | 4.87 | 8.50 | 8.41 | 5.49 | 6.54 | 3.84 |
| 30 Min | 5.65 | 6.77 | 6.73 | 6.90 | 6.61 | 3.93 |
| 45 Min | 5.15 | 7.67 | 7.81 | 6.03 | 5.57 | 3.53 |
| 60 Min | 4.96 | 8.07 | 7.97 | 6.19 | 5.04 | 3.61 |

### Tail Flick Test

In the Tail Flick test, the rats were allocated individually in a restrainer that is allowing their tail free. Then, the tail of each rat was placed over a 0.5 cm diameter infrared heat source. The tail flicking reaction time (how long it takes for the rat to flick its tail when placed over a heat source) of the rat's was recorded 1 hour after oral administration of the test compounds (administered by gavage) with the same dosages as the hot plate study. The animals were restrained in transparent Plexiglas tubes during the measurement. The tests were performed 3 times (with a 15 sec interval) at the 60 minutes time point, and the average of the measurements was used. For this experiment, the automated Ugo Basile Tail Flick instrument was used.

TABLE 5
The tail flick results in seconds, 1 hour after administration.

| | | FBP + | FBP + | | Paracetamol + | |
|---|---|---|---|---|---|---|
| | FBP | Propranolol | Metoprolol | Tramadol | Codeine | Placebo |
| 60 min | 4.95 sec | 10.06 sec | 9.71 sec | 5.81 sec | 6.46 sec | 2.21 sec |

### Evaluation of the Results:

In the hot plate test; Flurbiprofen administered alone had the lowest analgesic effect compared to all the other groups except the placebo group. FBP+Prop combined drug exhibited statistically significant analgesic activity in the hot plate model by increasing the reaction time of the rats to over 8.0 sec both at 15 min and 60 min after treatment in comparison to control (placebo) which was below 4 sec at both time points. FBP+Metoprolol combined drug exhibited highly similar and efficient results as far as analgesic effect is concerned. Tramadol produced a heat induced pain reaction time of less than 7 sec in the same test at all of the time points. Tramadol showed its peak of activity at 30 min. The paracetamol+codeine group produced a reaction time of less than 7 sec in the same test at all of the time points, with a peak of activity at 30 min as well. The superiority of the compositions of the present invention with a superior analgesic effect is evident by the surprising results achieved, where the co-administration of a NSAID (flurbiprofen) and beta blockers (propranolol (non-selective beta blocker) and metoprolol (beta-1 selective beta blocker)) and both had a peak activity at 15 minutes after administration and retain its superior analgesic activity throughout all the time points demonstrating a strong effect for pain relief. The only time point where FBP+Prop and FBP+Met were slightly lower efficacy than tramadol was at 30 minutes but there was no statistically significant difference between them. Most interestingly, pain tolerance time of FBP+Prop and FBP+Met treatment had their highest level at 15 min, while their 45 min and 60 min results were almost the same with 15 min. Thus the compositions of the present invention act both as a fast acting treatment modality and also as a longer acting modality for pain relief, which is also demonstrated in the tail flick test, which shows a significant difference between the FBP+Prop and FBP+Met groups and the others, including 2 well known opioids, 1 hour after the administration of the compositions.

1.-17. (canceled)

18. A method of treating pain, the method comprising administering to an individual in need of such treatment a pharmaceutical combination comprising:
a) an NSAID or a pharmaceutically acceptable salt or derivative thereof; and
b) a beta blocker or a pharmaceutically acceptable salt or derivative thereof,
wherein the pain is muscle pain, eye pain, orofacial pain, abdominal pain, gynecological pain, tissue or skin pain, inflammatory pain, pain resulting from inflammatory bowel disease, bone pain, back pain, gout pain, fibromylagia, allodynia, dysmenorrhea, headache, cluster headache, migraine pain, tendon pain, postoperative pain, post-stroke pain, cancer pain, dental pain, diabetic neuropathy, dysmenorrhea pain, stump pain, phantom pain, neuralgias, neuronitis, neuritis, AIDS-related neuropathy, MS-related neuropathy or spinal cord injury-related pain.

19. The method of claim 18, wherein the pain is muscle pain, back pain, post-operative pain, headache, dysmenorrhea, cluster headache, cancer pain or migraine pain.

20. The method of claim 18, wherein the pain is chronic pain.

21. The method of claim 18, wherein the pain is moderate to severe pain.

22. The method of claim 18, wherein the NSAID is selected from the group consisting of 2-Arylpropionic acids group including alminoprofen, benoxaprofencarprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid; salicylates group including alicylamide, salicyl salicylate, methyl salicylate, magnesium salicylate, faislamine, ethenzamide, diflunisal, choline magnesium salicylate, benorylate/benorilatem and amoxiprin, aspirin; arylalkanoic acids group including aceclofenac, acemetacin, alclofenac, bromfenac, diclofenac, etodolac, indometacin, nabumetone, oxametacin, proglumetacin, sulindac, and tolmetinor; and oxicams group including droxicam, lomoxicam, meloxicam, piroxicam, tenoxicam and pharmaceutically acceptable salts and derivatives thereof and their combinations.

23. The method of claim 18, wherein the NSAID is a non-selective COX inhibitor which is selected from the group consisting of ibuprofen, flurbiprofen, diclofenac, naproxen, meloxicam, piroxicam, acetyl salicylic acid, ketoprofen, dexketoprofen, ketorolac, lornoxicam, tenoxicam and pharmaceutically acceptable salts and derivatives thereof.

24. The method of claim 18, wherein the NSAID is selected from the group consisting of flurbiprofen, ibuprofen, diclofenac, dexketoprofen, piroxicam, tenoxicam, naproxen, meloxicam and pharmaceutically acceptable salts and derivatives thereof.

25. The method of claim 18, wherein the NSAID is selected from the group consisting of flurbiprofen, ibuprofen, diclofenac, naproxen, meloxicam and pharmaceutically acceptable salts and derivatives thereof.

26. The method of claim 18, wherein beta blocker is selected from the group consisting of acebutolol, atenolol, betaxolol, propranolol, bisoprolol, celiprolol, esmolol, metoprolol, nebivolol and pharmaceutically acceptable salts and derivatives thereof.

27. The method of claim 18, wherein beta blocker is selected from the group consisting of metoprolol, propranolol, nebivolol, atenolol, betaxolol, bisoprolol, carvedilol and pharmaceutically acceptable salts and derivatives thereof.

28. The method of claim 18, wherein the beta blocker is metoprolol, propranolol or pharmaceutically acceptable salts or derivatives thereof.

29. The method of claim 18, wherein the daily dose of beta blocker is between 4 to 80 mg.

30. The method of claim 18, wherein the daily dose of beta blocker is between 6 to 70 mg.

31. The method of claim 18, wherein the daily dose of beta blocker is between 8 to 60 mg.

32. The method of claim 18, wherein the combination is metoprolol in combination with flurbiprofen, metoprolol in combination with naproxen, propranolol in combination with flurbiprofen, propranolol in combination with naproxen, metoprolol in combination with diclofenac, metoprolol in combination with meloxicam, propranolol in combination with diclofenac, or propranolol in combination with meloxicam.

33. A method of treating an individual suffering from pain, the method comprising administering to an individual in need of such treatment a fixed unit oral dosage form of a pharmaceutical composition comprising,
a) an NSAID or a pharmaceutically acceptable salt thereof; and
b) a beta blocker or a pharmaceutically acceptable salt thereof,
wherein the pain is muscle pain, eye pain, orofacial pain, abdominal pain, gynecological pain, tissue or skin pain, inflammatory pain, pain resulting from inflammatory bowel disease, bone pain, back pain, gout pain, fibromylagia, allodynia, dysmenorrhea, headache, cluster headache, migraine pain, tendon pain, postoperative pain, post-stroke pain, cancer pain, dental pain, diabetic neuropathy, dysmenorrhea pain, stump pain, phantom pain, neuralgias, neuronitis, neuritis, AIDS-related neuropathy, MS-related neuropathy or spinal cord injury-related pain.

34. The method of claim 33, wherein the pain is muscle pain, back pain, post-operative pain, headache, dysmenorrhea, cluster headache, cancer pain or migraine pain.
